(19) United States
(12) Patent Application Publication
(10) Pub. No.: US 2008/0319200 A1
(43) Pub. Date: Dec. 25, 2008

(54) PROCESS FOR PREPARING OXAZOLINE-PROTECTED AMINODIOL COMPOUNDS USEFUL AS INTERMEDIATES TO FLORFENICOL

(75) Inventor: JAMES C. TOWSON, Flemington, NJ (US)

Correspondence Address: Intervet/Schering-Plough Animal Health;PATENT DEPARTMENT, PO BOX 318, 29160 Intervet Lane, MILLSBORO, DE, 19966-0318, US

(73) Assignee: Schering-Plough Animal Health Corp.

(21) Appl. No.: 12/128,146

(22) Filed: May 28, 2008

Related U.S. Application Data
(60) Provisional application No. 60/940,786, filed on May 30, 2007.

(51) Int. Cl. C07D 263/08

(52) U.S. Cl. 548/237

(57) ABSTRACT

A method of preparing oxazoline-protected aminodiol compounds is disclosed. These compounds are useful intermediates in processes for preparing Florfenicol and related compounds.

### TECHNICAL FIELD

The present invention relates to a process for preparing oxazoline-protected aminodiol compounds from ester amide and ester oxazoline compounds. These compounds are useful intermediates in the process for preparing Florfenicol and related compounds.

### BACKGROUND OF THE INVENTION

Florfenicol is a broad spectrum antibiotic of Formula I.

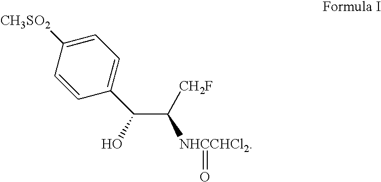

It has wide spread application in veterinary medicine for the treatment of both Gram positive and Gram negative bacteria as well as rickettsial infections. Florfenicol is also known as 2,2-dichloro-N-[(1S,2R)-1-(fluoromethyl)-2-hydroxy-2-[4-(methylsulfonyl)phenyl]ethyl}-acetamide or [R—(R*,S*)]-2,2-Dichloro-N-[1-(fluoromethyl)-2-hydroxy-2-[4-(methylsulfonyl)phenyl]ethyl]acetamide.

U.S. Pat. No. 5,663,361, the disclosure of which is incorporated herein by reference, describes the synthesis of Florfenicol intermediates of Formula II, where R₁ is phenyl or dichloromethyl, and their use in processes for making Florfenicol.

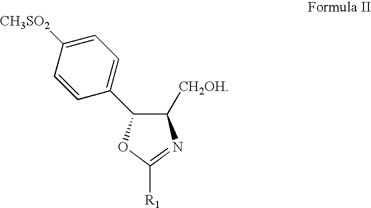

Japanese Patent Application No. JP1975148326(A), the publication by Clark et al., Synthesis, 1991, 891-894, and Chinese Patent No. CN1326926A, the disclosures of which are incorporated herein by reference, describe the preparation of the Florfenicol intermediate of Formula II, where R₁ is phenyl, from (2R,3S)-ethyl-2-amino-3-((4-(methylsulfonyl)phenyl)-3-hydroxy-propionate of Formula III.

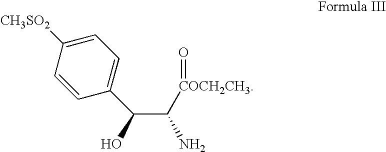

A major drawback of a process described above is that, when Florfenicol is a desired end-product, several additional steps must be taken to produce Florfenicol. Firstly, the compound of Formula II, when R₁ is phenyl, must be fluorinated; secondly, the phenyl oxazoline protecting group must be removed and the resulting equimolar benzoic acid waste disposed; thirdly, the resulting compound must be acylated to produce Florfenicol. This inefficient process results in high production and waste disposal costs. The present invention addresses these shortcomings.

Applicants have now surprisingly found significant processing advantages for forming the oxazoline-protected aminodiol compound of Formula IV from a compound of Formula V as a starting material, allowing for more efficient and cost-saving processes. The present invention thus has the advantage of being an efficient and economical process for preparing Florfenicol, its analogs, ester amide, ester oxazoline and oxazoline intermediates related thereto. The present invention is directed to oxazoline-protected aminodiol compounds and alternative methods of preparing useful intermediates included in the synthesis of Florfenicol.

### SUMMARY OF THE INVENTION

The present invention provides a process for preparing an oxazoline-protected aminodiol compound of Formula IV:

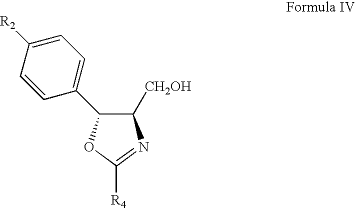

wherein:

R₂ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, or C₂₋₆ heterocyclic group;

and

R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₈ cyclohaloalkyl, C₃₋₈ cyclodihaloalkyl, C₃₋₈ cyclotrihaloalkyl, C₂₋₆ alkenyl, C₂₋₆ alkynyl, C₁₋₆ alkoxy, C₁₋₆ aralkyl, C₂₋₆ aralkenyl, C₂₋₆ heterocyclic, benzyl, or phenyl alkyl where the phenyl ring may be substituted by one or two halogens, C₁₋₆ alkyl or C₁₋₆ alkoxy; or an acid addition salt thereof.

In some embodiments, a process of the present invention includes the steps of reacting a compound of Formula V or an acid addition salt thereof:

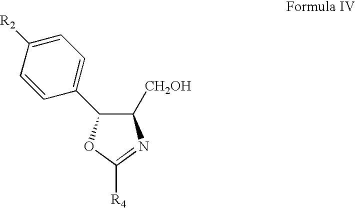

wherein:

R₂ is as defined above,

R₃ is hydrogen, C₁₋₆ alkyl, C₃₋₈ cycloalkyl, benzyl, phenyl or C₁₋₆ alkylphenyl; and the acid addition salt is the HCl, HNO₃, H₂SO₄, H₃PO₄, or acetic acid salt, in a vessel with an amide-promoting reagent in an amide-forming solvent with an amide-promoting compound to form the ester amide compound of Formula VI:

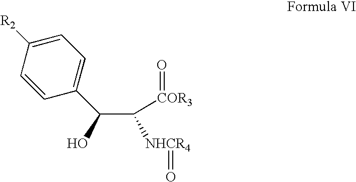

wherein R₂, R₃ and R₄ are as defined above.

In some embodiments, a process of the present invention continues by reacting the compound of Formula VI in a vessel, with isolation or without isolation (i.e., in situ), with an oxazoline-promoting reagent in an oxazoline-forming solvent in the presence of an oxazoline-promoting compound to form the ester oxazoline of Formula VI:

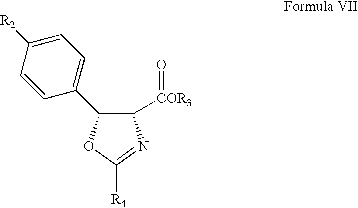

wherein R₂, R₃ and R₄ are as defined above and an inverted relative stereochemistry exists at the asymmetric benzylic carbon compared to that of the compound of Formula VI.

In some embodiments, a process of the present invention continues by reacting the compound of Formula VII in a vessel, with isolation or without isolation (i.e., in situ), with a chiral center-inverting base in a chiral center-inverting solvent to form the compound of Formula VIII:

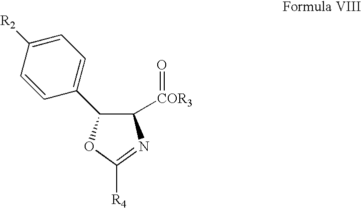

wherein R₂, R₃ and R₄ are as defined above and an inverted relative stereochemistry exists at the asymmetric α-carbonyl carbon compared to that of the compound of Formula VIII.

In some embodiments, a process of the present invention continues by reacting the compound of Formula VIII in a vessel, with isolation or without isolation (i.e., in situ), with a reducing agent in a reducing-promoting solvent to form the compound of Formula IV:

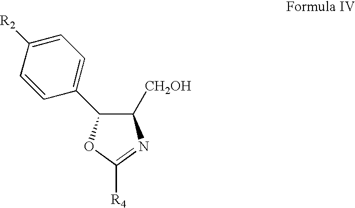

wherein R₂ and R₄ are as defined above.

In some embodiments, a process of the present invention continues with fluorinating the compound of Formula IV as described in U.S. Pat. Nos. 4,743,700, 4,876,352, 5,332,835, 5,382,673 and 5,567,844, the disclosures of which are incorporated herein by reference. In some embodiments, the process further continues by opening the oxazoline ring as described in U.S. Pat. No. 5,382,673 and Guangzhong et al. in J. Org. Chem. 62, 2996-98, (1997), the disclosures of which are incorporated herein by reference, to form Florfenicol and related compounds.

The present invention also provides a compound of Formula V or an acid addition salt thereof:

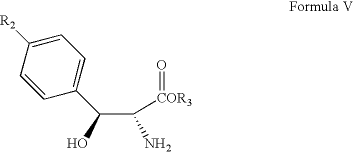

wherein R₂ and R₃ are as defined above, with the provisos that if R₂ is methylsulfonyl, then R₃ is not CH₃ or CH₂CH₃ and if the compound of Formula V is the acid addition salt, then the acid addition salt is the HCl, HNO₃, H₂SO₄, H₃PO₄, or acetic acid salt.

The present invention also provides a compound of Formula VI or an acid addition salt thereof:

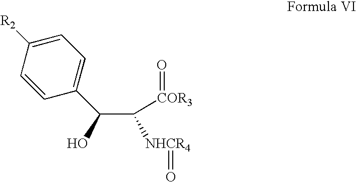

wherein R₂ is methylsulfonyl; R₃ is CH₃ or CH₂CH₃; and R₄ is CH₂Cl, CHCl₂, CCl₃, CH₂Br, CHBr₂, CBr₃ CH₂F, CHF₂, or CF₃ with the proviso that if the compound of Formula VI is the acid addition salt, then the acid addition salt is the HCl, HNO₃, H₂SO₄, H₃PO₄, or acetic acid salt.

The present invention also provides a compound of Formula VII or an acid addition salt thereof:

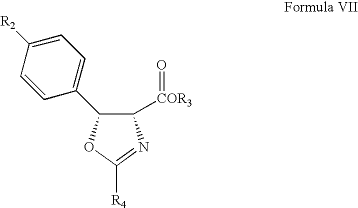

wherein R₂, R₃ and R₄ are as defined above, with the provisos that if R₂ is methylsulfonyl and R₄ is phenyl, then R₃ is not CH₃ or CH₂CH₃; and if the compound of Formula VI is the acid addition salt, then the acid addition salt is the HCl, HNO₃, H₂SO₄, H₃PO₄, or acetic acid salt.

The present invention further provides a compound of Formula VIII or anacid addition salt thereof:

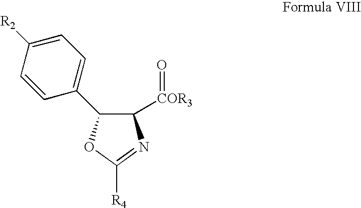

wherein R₂, R₃ and R₄ are as defined above, with the provisos that if R₂ is methylsulfonyl and R₄ is phenyl, then R₃ is not CH₃ or CH₂CH₃; and the compound of Formula VI is the acid addition salt, then the acid addition salt is the HCl, HNO₃, H₂SO₄, H₃PO₄, or acetic acid salt.

### DETAILED DESCRIPTION OF THE EMBODIMENTS

When used herein and in the appended claims, the terms listed below, unless otherwise indicated, will be used and are intended to be defined as indicated immediately below. Definitions for other terms can occur throughout the specification. It is intended that all terms used include the plural, active tense and past tense forms of a term.

The term “acetyl” means a CH₃CO— radical.

The term “alcoholic solvent” includes C₁ to C₁₀ monoalcohols such as methanol, ethanol, and mixtures thereof, C₂ to C₁₀ dialcohols such as ethylene glycol and C₁ to C₁₀ trialcohols such as glycerin. Alternatively, the term alcoholic solvent includes such alcohol admixed with any suitable cosolvent (i.e., a second solvent added to the original solvent, generally in small concentrations, to form a mixture that has greatly enhanced solvent powers due to synergism). Such cosolvents can include other solvents which are miscible with the alcoholic solvent such as C₄ to C₁₀ alkanes, aromatic solvents such as benzene, toluene, and xylenes, halobenzenes such as chlorobenzene, and ethers such as diethylether, tert-butylmethylether, isopropylether and tetrahydrofuran, or mixtures of any of the above cosolvents.

The term “alkyl” means a saturated straight or branched alkyl such as methyl, ethyl, propyl, or sec-butyl. Alternatively, the number of carbons in an alkyl can be specified. For example, “C₁₋₆ alkyl” means an “alkyl” as described above containing 1, 2, 3, 4, 5 or 6 carbon atoms.

The term “C₂₋₆ alkenyl” means an unsaturated branched or unbranched hydrocarbon group having at least one double carbon-carbon (—C≡C—) bond and containing 2, 3, 4, 5, or 6 carbon atoms. Example alkenyl groups include, without limitation, ethenyl, 1-propenyl, isopropenyl, 2-butenyl, 1,3-butadienyl, 3-pentenyl and 2-hexenyl, and the like.

The term “C₂₋₆ alkynyl” means an unsaturated branched or unbranched hydrocarbon group having at least one triple carbon-carbon (—C≡O—) bond and containing 2, 3, 4, 5, or 6 carbon atoms. Example alkynyl groups include, without limitation, ethynyl, 1-propynyl, 2-propynyl, 2-butynyl, 3-butynyl, 2-penten-4-ynyl, and the like.

The term “C₁₋₆ alkoxy” means an alkyl-O— group, where the term “alkyl” is defined herein. Example alkoxy groups include, without limitation, methoxy, ethoxy, propoxy (e.g., n-propoxy and isopropoxy), t-butoxy, and the like.

The term “aryl” means phenyl, or phenyl substituted by C₁ to C₆ alkyl or “halo”, where phenyl and halo are as defined herein.

The term “C₁₋₆ aralkyl” means a C₁₋₆ alkyl as defined herein substituted by an aryl group that is any radical derived from an aromatic hydrocarbon by the removal of a hydrogen atom.

The term “C₂₋₆ aralkenyl” means a C₂₋₆ alkenyl as defined herein substituted by an aryl group that is any radical derived from an aromatic hydrocarbon by the removal of a hydrogen atom.

The term “amide-promoting compound” refers to an acid or base that enhances, increase, accelerates or otherwise facilitates the reaction of the amide-promoting reagent with a free amine.

The term “amide-promoting reagent” refers to a reagent such that when reacted with a free amine will produce an amide wherein the carbonyl and substituent group attached to the carbonyl of the amide are from the amide-promoting reagent.

The term “amide-promoting solvent” is a solvent that enhances, increases, accelerates or otherwise facilitates the reaction between the amide-promoting reagent and the free amine.

The term “bromo” means the chemical element bromine.

“Substituted benzyl” means benzyl substituted by C₁ to C₆ alkyl or “halo”, where benzyl is the univalent radical C₆H₅CH₂, formally derived from toluene (i.e., methylbenzene).

The term “chloro” means the chemical element chorine.

The term “C₃₋₈ cycloalkyl” means a saturated cyclic hydrocarbon group (i.e., a cyclized alkyl group) containing 3, 4, 5, 6, 7 or 8 carbon atoms. Example cycloalkyl groups include, without limitation, cyclopropyl, cyclobutyl, cyclopentyl, cyclohexyl and the like.

The term “C₃₋₈ cyclohaloalkyl” means a C₃₋₈ cycloalkyl as defined herein substituted by halo as defined herein.

The term “C₃₋₈ cyclodihaloalkyl” means a C₃₋₈ cycloalkyl as defined herein substituted twice by halo as defined herein where the halo atoms can be the same or different.

The term “C₃₋₈ cyclotrihaloalkyl” means a C₃₋₈ cycloalkyl as defined herein substituted thrice by halo as defined herein where the halo atoms can be the same or different.

The term “C₂ to C₁₀ dialcohol” means an alcohol containing two hydroxyl groups and 2, 3, 4, 5, 6, 7, 8, 9 or 10 carbon atoms.

The term “C₁₋₆ dihaloalkyl” means a C₁₋₆ alkyl as defined herein substituted twice by halo as defined herein where the halo atoms can be the same or different.

The term “fluoro” means the chemical element fluorine.

The term “fluoromethylsulfonyl” means a CH₂FSO₂— radical.

The term “fluoromethylsulfoxy” means a CH₂FSO— radical.

The term “fluoromethylthio” means a CH₂FS— radical.

The term “halo” or “halogen” means fluoro, chloro, bromo or iodo.

“Haloalkyl” means an alkyl as described above wherein one or more hydrogens are replaced by halo as defined herein.

The term “halo substituted phenyl” means a phenyl as defined herein substituted by halo as defined herein.

The term “C₂₋₆ heterocyclic group” means a ring system radical where one or more of the ring-forming carbon atoms is replaced by a heteroatom, such as an oxygen, nitrogen, or sulfur atom, which include mono- or polycyclic (e.g., having 2 or more fused rings) ring systems as well as spiro ring systems. The ring system can contain 2, 3, 4, 5, or 6 carbon atoms and can be aromatic or non-aromatic.

“Iodo” means the chemical element iodine.

The term “methylsulfonyl” means a CH₃SO₂— radical.

The term “methylsulfoxy” means a CH₃SO— radical.

The term “methylthio” means a CH₃S— radical.

The term “C₁ to C₁₀ monoalcohol” means an alcohol containing one hydroxyl group and 1, 2, 3, 4, 5, 6, 7, 8, 9 or 10 carbon atoms.

The term “monosubstituted amino” means an —NH₂ radical where one of its hydrogen is substituted by another atom or radical.

The term “nitro” means a —NO₂ radical.

The term “oxazoline promoting compound” means a base that facilitates the formation and stability of the oxazoline ring formed by reaction of the oxazoline-promoting reagent with a α-hydroxy β-amide group.

The term “oxazoline-promoting reagent” means a reagent such that when reacted with an α-hydroxy β-amide group will produce an oxazoline ring, where the carbon and the substituent group of the carbon joining the oxygen of the hydroxyl function and the amine of the amide function in the oxazoline ring are derived from the “oxazoline-promoting reagent”.

The term “oxazoline-forming solvent” means a solvent that enhances, increases, accelerates of otherwise facilitates the reaction between the oxazoline-promoting reagent and the α-hydroxy β-amide group to form an oxazoline ring.

“Phenyl” means the monovalent radical C₆H₅— of benzene, which is the aromatic hydrocarbon C₆H₆.

The term “phenyl alkyl” means an alkyl as defined herein substituted by phenyl as defined herein.

The term “C₁ to C₁₀ trialcohol” means an alcohol containing three hydroxyl groups and 1, 2, 3, 4, 5, 6, 7, 8, 9 or 10 carbon atoms.

The term “C₁₋₆ trihaloalkyl” means a C₁₋₆ alkyl as defined herein substituted thrice by halo as defined herein where the halo atoms can be the same or different.

Throughout the specification and the appended claims, a given chemical formula or name shall encompass all stereo and optical isomers and racemates thereof, as well as mixtures in different proportions of the separate enantiomers, where such isomers and enantiomers exist, as well as pharmaceutically acceptable salts thereof and solvates thereof such as for instance hydrates. Isomers can be separated using conventional techniques, e.g., chromatography or fractional crystallization. The enantiomers can be isolated by separation of a racemic mixture, for example, by fractional crystallization, resolution or high-performance (or -pressure) liquid chromatography (HPLC). The diastereomers can be isolated by separation of isomer mixtures, for instance, by fractional crystallization, HPLC or flash chromatography. The stereoisomers also can be made by chiral synthesis from chiral starting materials under conditions which will not cause racemization or epimerization, or by derivatization, with a chiral reagent. The starting materials and conditions will be within the skill of one skilled in the art. All stereoisomers are included within the scope of the invention.

In one aspect, the present invention provides a process for preparing an oxazoline-protected aminodiol compound of Formula IV or an acid addition salt thereof:

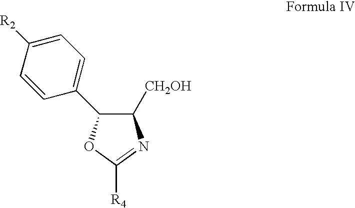

wherein:

R₂ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, or C₂₋₆ heterocyclic group; and

R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₈ cyclohaloalkyl, C₃₋₈ cyclodihaloalkyl, C₃₋₈ cyclotrihaloalkyl, C₂₋₆ alkenyl, C₂₋₆ alkynyl, C₁₋₆ alkoxy, C₁₋₆ aralkyl, C₂₋₆ aralkenyl, C₂₋₆ heterocyclic, benzyl, or phenyl alkyl wherein phenyl of the phenyl alkyl can be substituted by one or two halogens, C₁₋₆ alkyl, or C₁₋₆ alkoxy.

The oxazoline-protected aminodiol compounds of Formula IV of the present invention are useful intermediates in the formation of Florfenicol and related compounds.

In some embodiments, a process of the present invention includes the steps of:

a) reacting a compound of Formula V or an acid addition salt thereof:

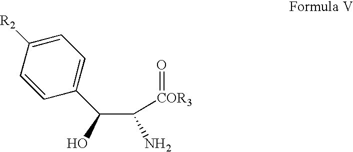

wherein:

R₂ is as defined above; and

R₃ is hydrogen, C₁₋₆ alkyl, C₃₋₈ cycloalkyl, benzyl, phenyl or C₁₋₆ alkylphenyl; with the proviso that if the compound of Formula V is the acid addition salt, then the acid addition salt is the HCl, HNO₃₁H₂SO₄, H₃PO₄, or acetic acid salt, in a vessel with an amide-promoting reagent in an amide-forming solvent with an amide-promoting compound to form an ester amide compound of Formula VI:

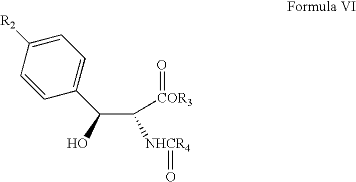

wherein R₂, R₃ and R₄ are as defined above;

b) reacting the compound of Formula VI in a vessel, with isolation or without isolation (i.e., in situ), with an oxazoline-promoting reagent in an oxazoline-forming solvent in the presence of an oxazoline-promoting compound to form an ester oxazoline compound of Formula VII:

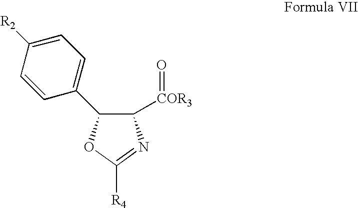

wherein R₂, R₃ and R₄ are as defined above and an inverted relative stereochemistry exists at the asymmetric benzylic carbon compared to that of the compound of Formula VI;

c) reacting the compound of Formula VII in a vessel, with isolation or without isolation (i.e., in situ), with a chiral center-inverting base in a chiral center-inverting solvent to form a compound of Formula VIII:

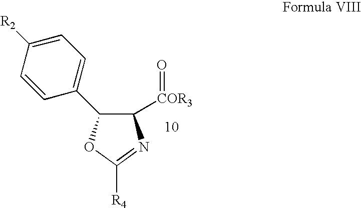

wherein R₂, R₃ and R₄ are as defined above and where an inverted relative stereochemistry exists at the asymmetric o-carbonyl carbon compared to that of the compound of Formula VII; and

d) reacting the compound of Formula VIII in a vessel, with isolation or without isolation (i.e. in situ), with a reducing agent in a reducing-promoting solvent to form a compound of Formula IV:

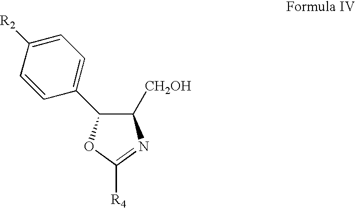

wherein R₂ and R₄ are as defined above.

In some embodiments, R₂ is methylthio, methylsulfoxy, or methylsulfonyl. In some embodiments, R₂ is methylsulfonyl.

In some embodiments, R₃ is methyl, ethyl, propyl, isopropyl, butyl, iso-butyl or pentyl. In some embodiments, R₃ is methyl or ethyl. In some embodiments, R₃ is ethyl.

In some embodiments, R₄ is CH₂Cl, CHCl₂, CCl₃, CH₂Br, CHBr₂, CBr₃, CH₂F, CHF₂, or CF₃. In some embodiments, R₄ is CH₂Cl, CHCl₂, or CCl₃. In some embodiments, R₄ is CHCl₂.

In some embodiments, the compound of Formula V (the starting material) is a compound of Formula Va or an acid addition salt thereof:

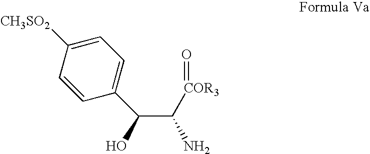

wherein R₃ is as defined above. In some embodiments, the compound of Formula Va is the acid addition salt. In some such embodiments, the acid addition salt is HCl.

In some embodiments, the compound of Formula V is a compound of Formula Vb or an acid addition salt thereof:

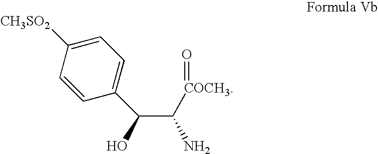

In some embodiments, the compound of Formula Vb is the acid addition salt. In some such embodiments, the acid addition salt is HCl.

In some embodiments, when Florfenicol is a desired end-product, the compound of Formula V is a compound of Formula Vc or the acid addition salt thereof:

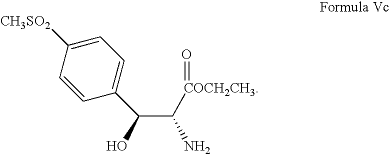

In some embodiments, the compound of Formula Vc is the acid addition salt. In some such embodiments, the acid addition salt is HCl.

As mentioned above, in some embodiments, the first part of a process of the present invention calls for reacting a compound of Formula V in a vessel with an amide-promoting reagent in an amide-forming solvent with an amide-promoting compound to form a compound of Formula VI:

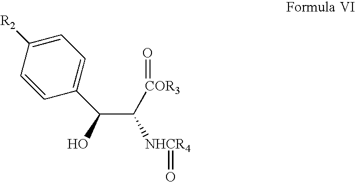

wherein R₂, R₃ and R₄ are as defined above.

As used herein, the term “vessel” or “reaction vessel” means a container known to those of ordinary skill in the art that is capable of holding the reactants while allowing the reaction step to proceed to completion. The size and type of vessel will, e.g., depend upon the size of the batch and the specific reactants selected.

A wide range of suitable amide-promoting reagents of the formula R₅COR₄, wherein R₄ is as defined above and R₅ is halo or C₁₋₆ alkoxy, can be used in carrying out a process of the present invention. In some embodiments, R₅ is C₁ or CH₃O and R₄ is CH₂Cl, CHCl₂, CCl₃, CH₂Br, CHBr₂, CBr₃, CH₂F, CHF₂, or CF₃. In some embodiments, R₄ is CH₂Cl, CHCl₂, or CCl₃. In some embodiments, R₄ is CHCl₂. In some embodiments, such as when Florfenicol is a desired end-product, the amide-promoting reagent is CH₃OCOCHCl₂ or ClCOCHCl₂. In some embodiments, the amide-promoting reagent is ClCOCHCl₂.

An amide-forming solvent useful in a process of the present invention can be one of many art-recognized solvents, for example and without limitation, methanol, ethanol, propanol, isopropanol, acetone, methylene chloride, ethyl acetate, tetrahydrofuran, ether, toluene or a mixture thereof. In some embodiments, the amide-forming solvent comprises methanol, ethanol, methylene chloride or a mixture thereof.

An amide-promoting compound useful in a process of the present invention can be one of many art-recognized compounds, for example and without limitation, potassium carbonate, potassium bicarbonate, sodium carbonate, sodium bicarbonate, trimethylamine, triethylamine, p-toluene sulfonic acid, methanesulfonic acid, acetic acid, hydrochloric acid, sulfuric acid, nitric acid, phosphoric acid or a mixture thereof. In some embodiments, the amide-promoting compound comprises triethylamine.

In some embodiments, the amide-promoting reagent and the compound of Formula V have a molar ratio between about 1:1 and about 3:1. In some embodiments, when the amide-promoting reagent is ClCOHCHCl₂, the molar ratio of ClCOCHCl₂ to the compound of Formula V is between about 1.2 and about 1.5 to about 1. In some embodiments, when the amide-promoting compound is triethylamine, the molar ratio of triethylamine to the compound of Formula V is between about 1.2 and about 1.5 to about 1. In some embodiments, when the amide-promoting compound is triethylamine, the molar ratio of triethylamine to an acid addition salt of the compound of Formula V is between about 2:1 and about 5:1. In some embodiments, the reaction step a) has a temperature between about minus 25° C. to about 25° C. In some embodiments, the reaction temperature is between about 0° C. to about 10° C.

In some embodiments, the compound of Formula VI is a compound of Formula VIa:

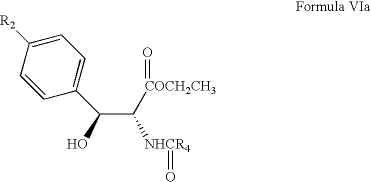

wherein R₂ and R₄ are as defined above.

In some embodiments, the compound of Formula VI is a compound of Formula VIb:

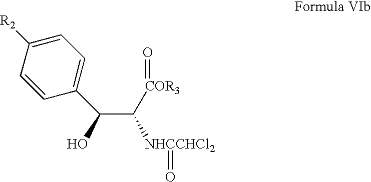

wherein R₂ and R₃ are as defined above.

In some embodiments, the compound of Formula VI is a compound of Formula VIc:

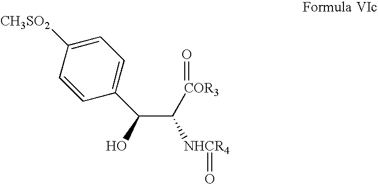

wherein R₃ and R₄ are as defined above.

In some embodiments, the compound of Formula VI is a compound of Formula VId:

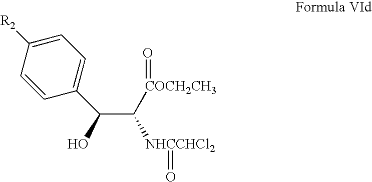

wherein R₂ is as defined above.

In some embodiments, the compound of Formula VI is a compound of Formula VIe:

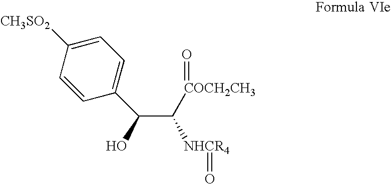

wherein R₄ is as defined above.

In some embodiments, the compound of Formula VI is a compound of Formula VIf:

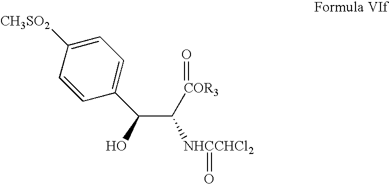

wherein R₃ is as defined above.

In some embodiments, such as when Florfenicol is a desired end-product, the compound of Formula VI is a compound of Formula VIg:

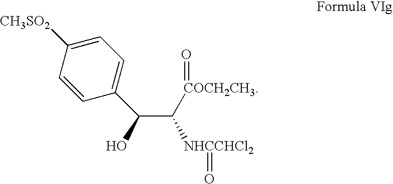

Once the amide ester compound of Formula VI is made, it is reacted, with isolation or without isolation (i.e., in situ), with an oxazoline-promoting reagent, such as and without limitation, thiony chloride, phosphorous trichloride, phosphorous pentachloride, phosphorous tribromide, phosphorous triiodide, phosphorous oxychloride, p-toluenesulfonyl chloride, p-bromosulfonyl chloride, p-nitrobenzenesulfonyl chloride, methanesulfonyl chloride, trifluoromethanesulfonyl chloride, nonafluorobutanesulfonyl chloride, 2,2,2-trifluoroethanesulfonyl chloride, or a mixture thereof, to form a compound of Formula VIII:

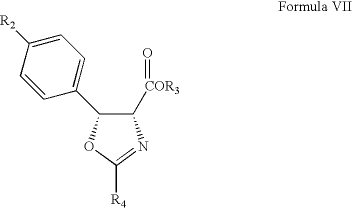

wherein R₂, R₃ and R₄ are as defined above and an inverted relative stereochemistry exists at the asymmetric benzylic carbon compared to that of the compound of Formula VI. In some embodiments, such as when Florfenicol is a desired end-product, the oxazoline-promoting reagent comprises thiony chloride.

An oxazoline-forming solvent useful in a process of the present invention can be one of many art-recognized solvents, for example and without limitation, methanol, ethanol, propanol, isopropanol, acetone, 1,2-dichloroethane, methylene chloride, chloroform, ethyl acetate, tetrahydrofuran, ether, toluene or a mixture thereof. In some embodiments, the oxazoline-forming solvent comprises methylene chloride, chloroform or a mixture thereof.

An oxazoline-promoting compound useful in a process of the present invention can be one of many art-recognized compounds, for example and without limitation, sodium carbonate, potassium carbonate, sodium bicarbonate, potassium bicarbonate, sodium hydroxide, potassium hydroxide, 1,4-diazabicyclo[2.2.2]octane, pyridine, trimethylamine, triethylamine or a mixture thereof. In some embodiments, the oxazoline-promoting reagent and the compound of Formula VI have a molar ratio between about 1:1 and about 6:1. In some embodiments, the molar ratio is about 2:1. In some embodiments, the oxazoline-promoting compound comprises triethylamine and the molar ratio of triethylamine to the oxazoline-promoting reagent is between about 1:1 and about 3:1. In some embodiments, the molar ratio is about 2:1.

In some embodiments, the reacting step b) of a process of the present invention has a temperature between about minus 25° C. to about 25° C. In some embodiments, the reaction temperature is between about 0° C. to about 10° C.

In some embodiments, the compound of Formula VIII is a compound of Formula VIIa:

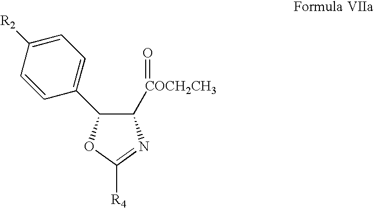

wherein R₂ and R₄ are as defined above.

In some embodiments, the compound of Formula VII is a compound of Formula VIIb:

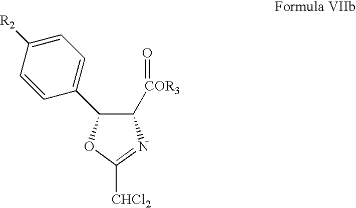

wherein R₂ and R₃ are as defined above.

In some embodiments, the compound of Formula VII is a compound of Formula VIIc:

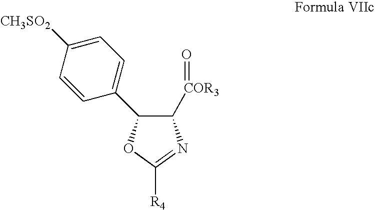

wherein R₃ and R₄ are as defined above.

In some embodiments, the compound of Formula VII is a compound of Formula VIId:

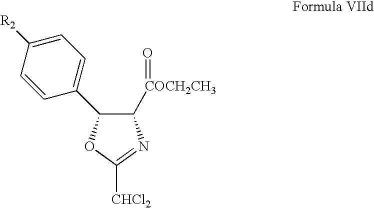

wherein R₂ is as defined above.

In some embodiments, the compound of Formula VII is a compound of Formula VIIe:

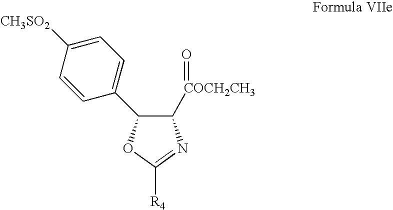

wherein R₄ is as defined above.

In some embodiments, the compound of Formula VII is a compound of Formula VIIf:

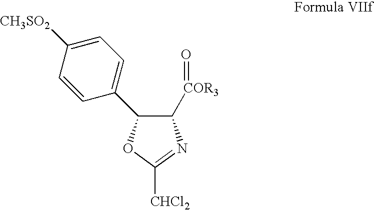

wherein R₃ is as defined above.

In some embodiments, such as when Florfenicol is a desired end-product, the compound of Formula VII is a compound of Formula VIIg:

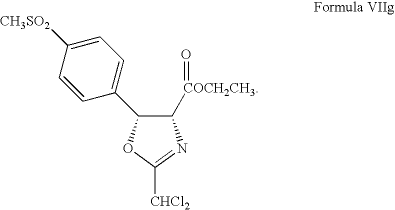

Once the ester oxazoline compound of Formula VII is made, it is reacted, with isolation or without isolation (i.e., in situ), with a chiral center-inverting base, such as and without limitation, sodium methoxide, sodium ethoxide, potassium methoxide, potassium ethoxide, sodium hydroxide, potassium hydroxide or a mixture thereof, to form a compound of Formula VIII:

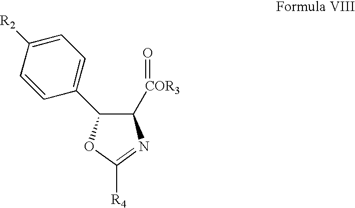

wherein R₂, R₃ and R₄ are as defined above and where an inverted relative stereochemistry exists at the asymmetric α-carbonyl carbon compared to that of the compound of Formula VII. As used herein, the term “chiral center-inverting base” refers to a base that will abstract a hydrogen from a chiral α-carbonyl carbon causing the α-carbonyl carbon relative stereochemical configuration to be inverted or opposite to that of its original stereochemical configuration.

The term, “chiral center-inverting solvent,” as used herein, refers to a solvent that enhances, increases, accelerates or otherwise facilitates the inversion of relative stereochemistry by the chiral center-inverting base at an α-carbonyl carbon. A chiral center-inverting solvent useful in a process of the present invention can be one of many art-recognized solvents, such as but without limitation, methanol, ethanol, propanol, isopropanol, acetone, methylene chloride, ethyl acetate, tetrahydrofuran, ether, toluene or a mixture thereof. In some embodiments, the chiral center-inverting solvent comprises methanol, ethanol, methylene chloride or a mixture thereof.

In some embodiments, the compound of Formula VIII is a compound of Formula VIIIa:

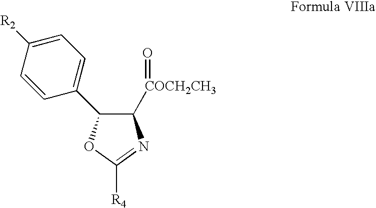

wherein R₂ and R₄ are as defined above.

In some embodiments, the compound of Formula VIII is a compound of Formula VIIIb:

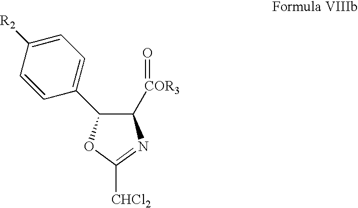

wherein R₂ and R₃ are as defined above.

In some embodiments, the compound of Formula VIII is a compound of Formula VIIIc:

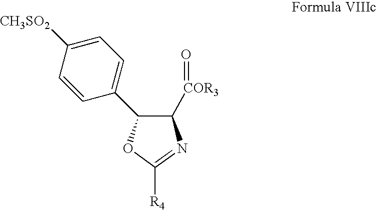

wherein R₃ and R₄ are as defined above.

In some embodiments, the compound of Formula VIII is a compound of Formula VIIId:

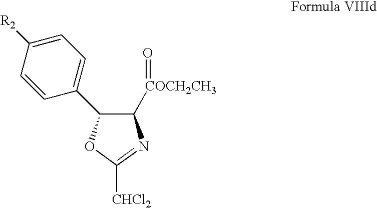

wherein R₂ is as defined above.

In some embodiments, the compound of Formula VIII is a compound of Formula VIIIe:

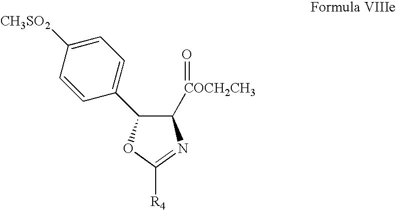

wherein R₄ is as defined above.

In some embodiments, the compound of Formula VIII is a compound of Formula VIIIf:

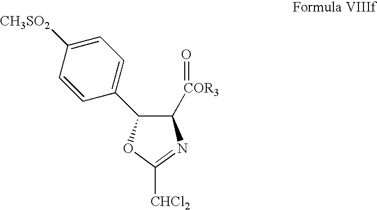

wherein R₃ is as defined above.

In some embodiments, such as when Florfenicol is a desired end-product, the compound of Formula VIII is a compound of Formula VIIIg:

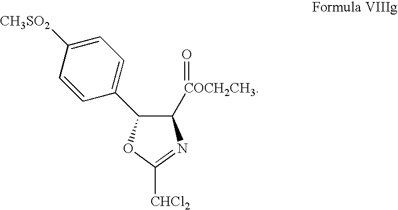

Once the compound of Formula VIII is made it is reacted, with isolation or without isolation (i.e., in situ), with a reducing agent in a reducing-promoting solvent to form a compound of Formula IV:

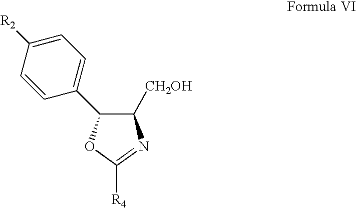

wherein R₂ and R₄ are as defined above.

As used herein, the term “reducing agent” refers to a reagent that facilitates the loss of an oxygen atom from a compound with a gain of electrons by the compound or the decrease in oxidation number (oxidation state) of a compound. A wide range of suitable reducing agents can be employed in carrying out a process of the present invention. A non-limiting list of suitable reducing agents include NaBH₄, KBH₄, Ca(BH₄)₂, LiBH₄ and a mixture thereof. In some embodiments, the reducing agent comprises KBH₄, NaBH₄, or a mixture thereof. In some embodiments, the reducing agent comprises KBH₄.

As used herein, the term “reducing-promoting solvent” refers to a solvent that facilitates the loss of an oxygen atom from a compound with a gain of electrons by the compound or the decrease in oxidation number (oxidation state) of a compound. A reducing-promoting solvent of a process of the present invention can be one of many art-recognized solvents, for example and without limitation, water, methanol, ethanol, propanol, isopropanol, butanol, pentanol and a mixture thereof.

In some embodiments, the reducing-promoting solvent comprises water, methanol, ethanol or a mixture thereof. In some embodiments, the reducing-promoting solvent comprises methanol.

In some embodiments, the reducing agent and the compound of Formula VIII have a molar ratio between about 1:1 and about 2:1. In some embodiments, when the reducing agent is KBH₄, the molar ratio of KBH₄ to the compound of Formula VIII is about 1.5:1. In some such embodiments, the reducing-promoting solvent comprises methanol.

In some embodiments, the reacting step d) can be carried out at a temperature of about 30° C. to about 80° C. in about 8 hours. In some embodiments, the temperature is less than about 60° C. and the reacting step is substantially complete in less than about 6 hours.

In some embodiments, such as when anhydrous conditions are desired, the reducing agent comprise, e.g., LiAlH₄, NaAlH₄, or a mixture thereof. In such embodiments, the reducing-promoting solvent comprises, erg., ether, tetrahydrofuran or a mixture thereof.

In some embodiments, the compound of Formula IV is a compound of Formula IVa:

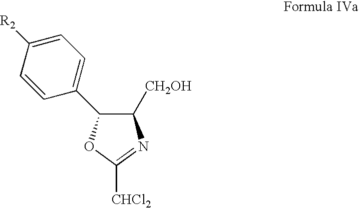

wherein R₂ is as defined above.

In some embodiments, the compound of Formula IV is a compound of Formula IVb:

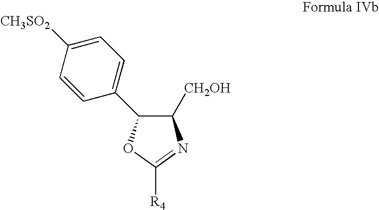

wherein R₄ is as defined above.

In some embodiments, such as when Florfenicol is a desired end-product, the compound of Formula IV is a compound of Formula IVc:

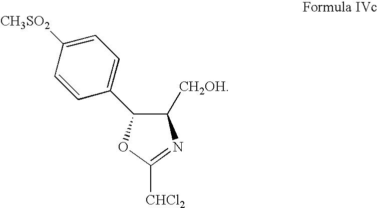

Once the compound of Formula IV has been prepared, one can use this compound as an intermediate for preparing Florfenicol and related compounds. Therefore, in some embodiments, a process of the present invention then continues with fluorinating the compound of Formula IV with a fluorinating agent, with isolation or without isolation (i.e., in situ), in the presence of an organic solvent to obtain the compound of Formula IX:

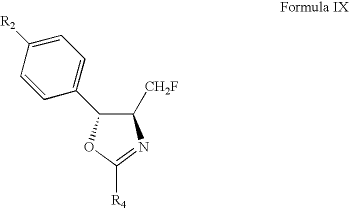

wherein R₂ and R₄ are as defined above.

Suitable fluorinating agents for a process of the present invention include, without limitation, sodium fluoride, potassium fluoride, cesium fluoride, tetrabutylammonium fluoride, 1,1,2,2,3,3,4,4,4-nonafluoro-1-butanesulfonyl fluoride, chloromethyl-4-fluoro-1,4-diazoniabicyclo[2.2.2]octane bis-(tetrafluoroborate), N-(2-chloro-1,1,2-trifluoroethyl)diethylamine, N-(2-chloro-1,1,2-trifluoroethyl)dimethylamine, N-(2-chloro-1,1,2-trifluoroethyl)dipropylamine, N-(2-chloro-1,1,2-trifluoroethyl)pyrrolidine, N-(2-chloro-1,12-trifluoroethyl)-2-methylpyrrolidine, N-(2-chloro-1,1,2-trifluoroethyl)-4-methylpiperazine, N-(2-chloro-1,1,2-trifluoroethyl)-morpholine, N-(2-chloro-1,1,2-trifluoroethyl)piperidine, 1,1,2,2-tetrafluoroethyl-N,N-dimethylamine, (Diethylamino) sulfur trifluoride, Bis-(2-methoxyethyl)aminosulfur trifluoride, N,N-diethyl-1,1,2,3,3,3-hexafluoro-1-propanamine (Ishikawa Reagent) and a mixture thereof.

In some embodiments, the fluorinating agent comprises N,N-diethyl-1,1,2,3,3,3-hexafluoro-1-propanamine. In some embodiments, the fluorinating agent, such as N,N-diethyl-1,1,2,3,3,3-hexafluoro-1-propanamine, and the compound of Formula IV have a molar ratio between about 1:1 and about 2:1. In some embodiments, the molar ratio of the N,N-diethyl-1,1,2,3,3,3-hexafluoro-1-propanamine to the compound of Formula IV is about 1.5:1.

In some embodiments, the fluorinating step is carried out at a temperature of about 80° C. to about 110° C. and at a pressure of about 60 psi.

In some embodiments, the organic solvent used during the fluorinating step comprises 1,2-dichloroethane, methylene chloride, chloroform, chlorobenzene, a chlorinated hydrocarbon or a mixture thereof. In some embodiments, the organic solvent comprises methylene chloride.

In some embodiments, the compound of Formula IX corresponds to a compound of Formula IXa:

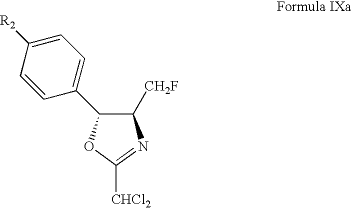

wherein R₂ is as defined above.

In some embodiments, the compound of Formula IX corresponds to a compound of Formula IXb:

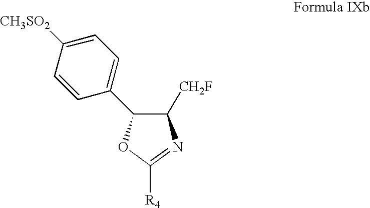

wherein R₄ is as defined above.

In some embodiments, such as when Florfenicol is a desired end-product, the compound of Formula IX corresponds to a compound of Formula IXc:

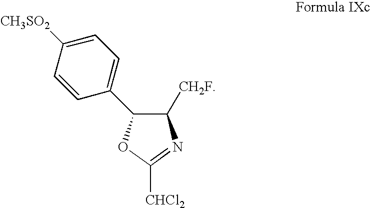

After the compound of Formula IX has been prepared, it can be hydrolyzed, with isolation or without isolation (tie, in situ), with water and an acid catalyst or a basic catalyst to form a compound of Formula X:

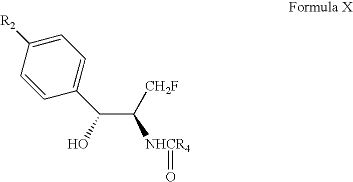

wherein R₁ and R₄ are as defined above

A wide range of acid catalysts can be employed in carrying out a process of the present invention. A non-limiting list of suitable acid catalysts includes inorganic acids, such as dilute aqueous hydrochloric acid, sulfuric acid, nitric acid, phosphoric acid and a mixture thereof, as well as organic acids, such as acetic acid, trifluoroacetic acid, methanesulfonic acid, p-toluene sulfonic acid and a mixture thereof. In some embodiments, the acid catalyst is a mixture of at least one inorganic acid and at least one organic acid. In some embodiments, the acid catalyst comprises p-toluene sulfonic acid.

A wide range of basic catalysts can be employed in carrying out of a process of the present invention. A non-limiting list of suitable basic catalysts includes inorganic bases, such as LiOH, NaOH, KOH, Li₂CO₃, Na₂CO₃, K₂CO₃, NH₄OH and a mixture thereof, as well as organic bases such as sodium methoxide, sodium ethoxide, potassium methoxide, potassium ethoxide and a mixture thereof. In some embodiments, the basic catalyst is a mixture of at least one inorganic acid and at least one organic acid. In some embodiments, the basic catalyst comprises NH₄OH.

In some embodiments, the hydrolyzing step is carried out with the compound of Formula IX and the acid catalyst or the basic catalyst in an organic solvent, water or a mixture of an organic solvent and water. A non-limiting list of organic solvents useful in the hydrolyzing step include acetone, methanol, ethanol, propanol, isopropanol, methylene chloride, ethyl acetate, tetrahydrofuran and a mixture thereof. In some embodiments, the organic solvent comprises isopropanol, methylene chloride or a mixture thereof. In some embodiments, the mixture of an organic solvent and water comprises methylene chloride. In some embodiments, about 0.5 to about 3 molar equivalents of water are used for each mole of the compound of Formula IX. In some embodiments, about 1 to about 2 molar equivalents of water are used for each mole of the compound of Formula IX.

The hydrolyzing step of a process of the present invention can be carried out at a temperature up to about 100° C. That is to say, hydrolysis is performed at a temperature less than or equal to about 100° C. In some embodiments, the temperature is less than about 30° C.

In some embodiments of a process of the present invention, the hydrolyzing step further comprises heating the compound of Formula IX with the acid catalyst or the basic catalyst in a mixture of an organic solvent and water at a temperature less than about 100° C.

Other suitable hydrolyzing steps will be apparent to those of ordinary skill in the art.

In some embodiments, the compound of Formula X corresponds to a compound of Formula Xa:

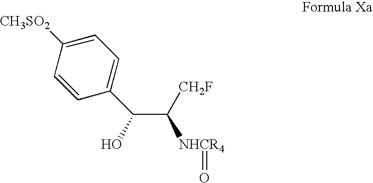

wherein R₄ is as described above.

In some embodiments, the compound of Formula X corresponds to a compound of Formula Xb:

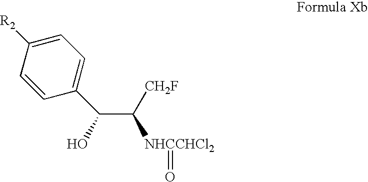

wherein R₂ is as described above.

In some embodiments, such as when Florfenicol is a desired end-product, the compound of Formula X corresponds to Florfenicol of Formula I:

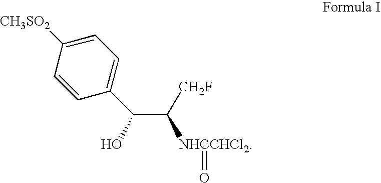

In some embodiments of a process of the present invention, the resultant amide ester compound of Formula VI, the resultant ester oxazoline compound of Formula VII, the resultant compound of Formula VIII, the resultant compound of Formula IV, the resultant fluorinated compound of Formula IX, the resultant hydrolyzed compound of Formula X, or any combination thereof, is isolated. In some embodiments, the resultant compound or any combination thereof is not isolated (i.e., is in situ).

After the compound of Formula X has been prepared, the compound of Formula X optionally can be purified with a mixture of a C₁₋₁₀ alkyl monoalcohol, a C₁₋₁₀ alkyl dialcohol or a C₁₋₁₀ alkyl trialcohol and water to form the pure compound of Formula X. A non-limiting list of C₁₋₁₀ monoalcohols includes methanol, ethanol, propanol, isopropanol, butanol, sec-butanol, t-butanol, pentanol and a mixture thereof. A non-limiting list of C₁₋₁₀ dialcohols includes ethylene glycol, propylene glycol, butylene glycol and a mixture thereof. A non-limiting example of a C₁₋₁₀ trialcohol is glycerin.

in some embodiments of a process of the present invention, the C₁₋₁₀ monoalcohol for the purifying step comprises isopropanol. In some embodiments of a process of the present invention, the C₁₋₁₀ dialcohol of the purifying step comprises propylene glycol. In some embodiments of a process of the present invention, the C₁₋₁₀ trialcohol of the purifying step comprises glycerin. In some embodiments, when Florfeniocl is a desired end-product, the mixture of alcohol and water comprises at least one C₁₋₁₀ monoalcohol. In some such embodiments, the at least one C₁₋₁₀ monoalcohol is isopropanol.

In some embodiments, the alcohol (such as isopropanol) and water are present in a ratio between about 1:5 and about 5:1. In some embodiments, the ratio of alcohol to water is about 1:1. In some embodiments, the alcohol comprises isopropanol and the ratio of the isopropanol to water mixture is about 1:1. In some embodiments, the compound of Formula X and the about 1:1 isopropanol and water mixture have a weight to volume ratio between about 1:1 and about 10:1. In some embodiments, the weight to volume ratio of the compound of Formula X to the about 1:1 isopropanol and water mixture is about 1:4.6.

In some embodiments of the purifying step of a process of the present invention, the compound of Formula X is dissolved in an about 1:1 isopropanol and water mixture, where the compound of Formula X and the about 1:1 isopropanol and water mixture have a weight to volume ratio of about 1:4.6, and heated to the reflux point of the mixture. The resultant solution is clarified by filtration with active carbon and a filter aid, then cooled to about 10° C. to about 30° C. to obtain crystallized compound of Formula X that is pure. As used herein, the terms “pure” or “purified” means reduced levels of impurities and improved color compared to unpurified compound. In some embodiments, the solution is cooled to about 20° C. to about 25° C. to crystallize the purified compound of Formula X from the solution. In some embodiments, the purified compound of Formula X crystallized from the solution is Florfenicol.

In another aspect, the present invention provides a compound of Formula V or an acid addition salt thereof:

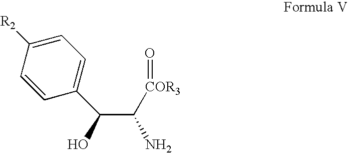

wherein R₂ and R₃ are as defined above, with the provisos that if R₂ is methylsulfonyl, then R₃ is not CH₃ or CH₂CH₃ and if the compound of Formula V is the acid addition salt, then the acid addition salt is the HCl, HNO₃, H₂SO₄, H₃PO₄, or acetic acid salt.

In another aspect, the present invention provides a compound of Formula VI or an acid addition salt thereof:

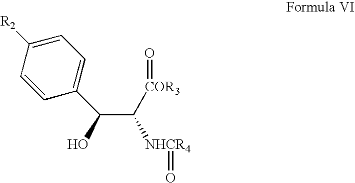

wherein R₂ methylsulfonyl; R₃ is CH₃ or CH₂CH₃; and R₄ is CH₂Cl, CHCl₂, CCl₃, CH₂Br, CHBr₂, CBr₃, CH₂F, CHF₂, or CF₃ with the proviso that if the compound of Formula VI is the acid addition salt, then the acid addition salt is the HCl, HNO₃, H₂SO₄, H₃PO₄, or acetic acid salt. In some such embodiments, R₄ is CH₂Cl.

In another aspect, the present invention provides a compound of Formula VII or an acid addition salt thereof:

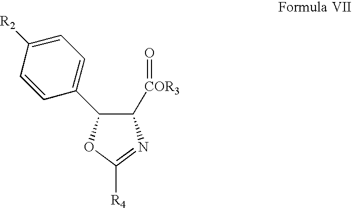

wherein R₂, R₃ and R₄ are as defined above, with the provisos that if R₂ is methylsulfonyl and R₄ is phenyl, then R₃ is not CH₃ or CH₂CH₃ and if the compound of Formula VI is the acid addition salt, then the acid addition salt is the HCl, HNO₃, H₂SO₄, H₃PO₄, or acetic acid salt. In some such embodiments, R₂ is methylsulfonyl, R₃ is CH₃ or CH₂CH₃; and R₄ is CHCl₂.

In another aspect, the present invention provides a compound of Formula VIII or an acid addition salt thereof:

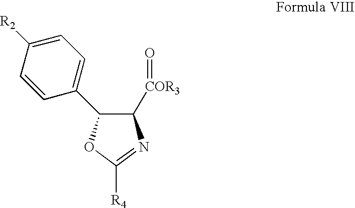

wherein R₂, R₃ and R₄ are as defined above, with the provisos that if R₂ methylsulfonyl and R₄ is phenyl, then R₃ is not CH₃ or CH₂CH₃ and if the compound of Formula VI is the acid addition salt, then the acid addition salt is the HCl, HNO₃, H₂SO₄, H₃PO₄, or acetic acid salt. in some such embodiments, wherein R₂ is methylsulfonyl, R₃ is CH₃ or CH₂CH₃; and R₄ is CHCl₂.

### EXAMPLES

The following hypothetical preparative examples are representative examples of a process and compounds of the present invention. While the present invention has been described with specificity in accordance with certain embodiments of the present invention, the following examples further serve only to exemplify and illustrate the present invention and are not intended to limit or restrict the effective scope of the present invention.

### Example 1

### Preparation of (2R,3S) Ethyl 2-(dichloroacetamido)-3-[4-(methylsulfonyl)phenyl]-3-hydroxy-propanoate (Compound VIg)

(2R,3S) Ethyl 2-amino-3-[4-(methylsulfonyl)phenyl]-3-hydroxy-propanoate (Compound II) (5 g, 0.01740 moles) in methanol (about 75 mL) containing triethylamine (about 2.1 g, 0.0210 moles) can react with dichloroacetyl chloride (about 3.1 g, 0.0210 moles) at about 0° C. to about 10° C. Addition of methylene chloride and water, separation of the organic layer, washing of the organic layer with water then evaporation of the solvent and drying can yield (2R,3S) ethyl 2-(dichloroacetamido)-3-[4-(methylsulfonyl)phenyl]-3-hydroxy-propanoate (Compound VIg).

### Example 2

### Preparation of Ethyl 2-(dichloromethyl)-4,5-dihydro-5(R)-[4-(methylsulfonyl)phenyl]-4(R)-oxazolecarboxylate (Compound VIIg)

(2R,3S) Ethyl 2-(dichloroacetamido)-3-[4-(methylsulfonyl)phenyl]-3-hydroxy-propanoate (Compound VIg) (about 5 g, 0.0126 moles) in chloroform (about 50 mL) can react with thionyl chloride (about 3.0 g, 0.0252 moles) at about 0° C. to about 10° C. Addition of triethylamine (about 5.1 g, 0.0504 moles) and water (about 100 mL), separation of the layers, washing of the organic layer with additional water, evaporation of the solvent and then drying can yield ethyl 2-(dichloromethyl)-4,5-dihydro-5(R)-[4-(methylsulfonyl)phenyl]-4(R)-oxazolecarboxylate (Compound VIIg).

### Example 3

### Preparation of Ethyl 2-(dichloromethyl)-4,5-dihydro-5(R)-[4-(methylsulfonyl)phenyl]-4(S)-oxazolecarboxylate (Compound VIIIg)

Ethyl 2-(dichloromethyl)-4,5-dihydro-5(R)-[4-(methylsulfonyl)phenyl]-4(R)-oxazolecarboxylate (Compound VIIg) (about 5 g, 0.0131 moles) in methanol (about 50 mL) containing sodium methoxide (about 0.7 g, 0.0131 moles) can epimerize. Then neutralization with hydrochloric acid, addition of methylene chloride (about 200 mL), extraction of the organic layer with water, evaporation of the solvent, filtration of the resulting solids and drying can yield ethyl 2-(dichloromethyl)-4,5-dihydro-5(R)-[4-(methylsulfonyl)phenyl]-4(S)-oxazolecarboxylate (Compound VIIIg).

### Example 4

### Preparation of (4R,5R)-2-(Dichloromethyl)-4,5-dihydro-5-[4-(methylsulfonyl)phenyl]-4-oxazolemethanol (Compound IVc)

Ethyl 2-(dichloromethyl)-4,5-dihydro-5(R)-[4-(methylsulfonyl)phenyl]-4(S)-oxazolecarboxylate (Compound VIIIg) (about 5 g, 0.0131 moles) in methanol (about 50 mL) can react with potassium borohydride (about 1.1 g, 0.0204 moles) over about 6 hours while maintaining the temperature below about 60° C. Addition of about 1N HCl and water, filtration of the resulting solids, washing with water and drying can yield (4R,5R)-2-(dichloromethyl)-4,5-dihydro-5-[4-(methylsulfonyl)phenyl]-4-oxazolemethanol (Compound IVc).

### Example 5

### Preparation of (4R,5R)-2-(dichloromethyl)-4,5-dihydro-5-[4-(methylsulfonyl)phenyl]-4-oxazolemethanol (Compound IVc)

Step 1: (2R,3S) Ethyl 2-amino-3-[4-(methylsulfonyl)phenyl]-3-hydroxy-propanoate (Compound II) (about 5g, 0.01740 moles) in methanol (about 75 mL) containing triethylamine (about 2.1 g, 0.0210 moles) can react with dichloroacetyl chloride (about 3.1 g, 0.0210 moles) at about 0° C. to about 10° C. to form (2R,3S) Ethyl 2-(dichloroacetamido)-3-[4-(methylsulfonyl)phenyl]-3-hydroxy-propanoate (Compound VIg), which is used without isolation in the next step.

Step 2: Evaporation of the methanol and replacement with methylene chloride, cooling to about 0° C. to about 10° C., addition of thionyl chloride (about 4.1 g, 0.0348 moles) with stirring for about 2 hours followed by addition of a mixture of ice and water, separation of the organic layer, washing with saturated NaHCO₃ and water can yield ethyl 2-(dichloromethyl)-4,5-dihydro-5(R)-[4-(methylsulfonyl)phenyl]-4(R)-oxazolecarboxylate (Compound VIIg), which is used without isolation in the next step.

Step 3: Evaporation of the methylene chloride and replacement with methanol, addition of sodium methoxide (about 0.9 g, 0.0174 moles) and neutralization with hydrochloric acid can yield ethyl 2-(dichloromethyl)-4,5-dihydro-5(R)-[4-(methylsulfonyl)phenyl]-4(S)-oxazolecarboxylate (Compound VIIIg) in situ.

Step 4: To Compound VIIIg in situ, potassium borohydride (about 1.4 g, 0.0261 moles) can be added with stirring over about 6 hours while maintaining the temperature below about 60° C. Then, addition of about 1N HCl and water, filtration of the resulting solids, washing with water and drying can yield (4R,5R)-2-(dichloromethyl)-4,5-dihydro-5-[4-(methylsulfonyl)phenyl]-4-oxazolemethanol (Compound IVc).

### Example 6

### Preparation of Florfenicol (Compound I)

(4R,5R)-2-(Dichloromethyl)-4,5-dihydro-5-[4-(methylsulfonyl)phenyl]-4-oxazolemethanol (Compound IVc) (about 5 g, 0.0148 moles) in methylene chloride containing N,N-diethyl-1,1,2,3,3,3-hexafluoro-1-propanamine (about 5 g, 0.0224 moles) can react at about 95° C. to about 100° C. to produce (4S,5R)-2-(dichloromethyl)-4-(fluoromethyl)-4,5-dihydro-5-[4-(methylsulfonyl)phenyl]-oxazole (Compound IXc) in situ. Cooling to below about 25° C., addition of water (about 0.4 g 0.0222 moles) and ammonium hydroxide (about 0.0237 moles), filtration of the resulting solids, washing with isopropanol and water and then drying can yield Florfenicol (Compound I).

### Example 7

### Purification of Florfenicol

Florfenicol (Compound I) of Example 6 (about 25 g, 0.0700 moles) can be dissolved in water (about 60 mL) and isopropanol (about 60 mL) at reflux to provide a mixture. Following addition of charcoal, clarification by filtration, cooling to about 20° C. to about 25° C., filtration of the solids, washing with about 1:1 water/isopropanol (about 20 mL) then drying, the mixture can yield pure Florfenicol (Compound I).

It must be noted that, as used herein and in the appended claims, the singular forms “a”, “an”, and “the” include plural references, unless the context clearly dictates otherwise. It is intended that each of the patents, patent applications, technical articles and reports, government, trade and industry publications, printed publications, including books and any of the aforementioned publications, mentioned in this patent document be hereby incorporated by reference in its entirety.

As those skilled in the art will appreciate, numerous changes and modifications can be made to the embodiments of the invention without departing from the spirit of the invention. It is intended that all such variations fall within the scope of the invention.

What is claimed is:

1. A process for preparing an oxazoline-protected aminodiol compound of Formula IV or an acid addition salt thereof: 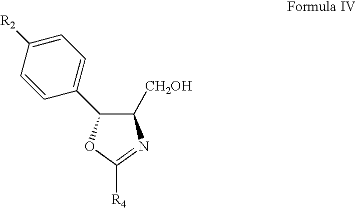
wherein:
R₂ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, or C₂₋₆ heterocyclic group; and
R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂ CF₃, C₃₋₈ cycloalkyl, C₃₋₈ cyclohaloalkyl, C₃₋₈ cyclodihaloalkyl, C₃₋₈ cyclotrihaloalkyl, C₂₋₆ alkenyl, C₂₋₆ alkynyl, C₁₋₆ alkoxy, C₁₋₆ aralkyl, C₂₋₆ aralkenyl, C₂₋₆ heterocyclic, benzyl, or phenyl alkyl wherein phenyl of the phenyl alkyl can be substituted by one or two halogens, C₁₋₆ alkyl, or C₁₋₆ alkoxy;
the process comprising the steps of:
a) reacting a compound of Formula V or an acid addition salt thereof: 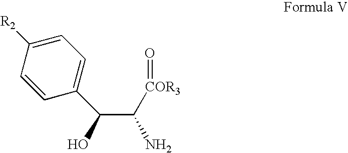
wherein:
R₂ is as defined above; and
R₃ is hydrogen, C₁₋₆ alkyl, C₃₋₈ cycloalkyl, benzyl, phenyl or C₁₋₆ alkylphenyl;
with the proviso that if the compound of Formula V is the acid addition salt, then the acid addition salt is the HCl, HNO₃, H₂SO₄, H₃PO₄, or acetic acid salt,
with an amide-promoting reagent in an amide-forming solvent with an amide-promoting compound to form an ester amide compound of Formula VI: 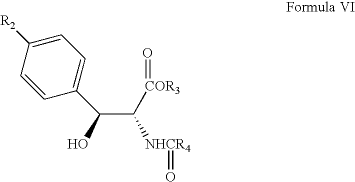
wherein R₂, R₃ and R₄ are as defined above;
b) reacting the compound of Formula VI with an oxazoline-promoting reagent in an oxazoline-forming solvent in the presence of an oxazoline-promoting compound to form an ester oxazoline compound of Formula VII: 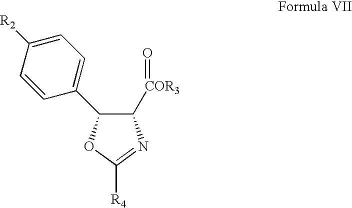
wherein R₂, R₃ and R₄ are as defined above and an inverted relative stereochemistry exists at the asymmetric benzylic carbon compared to that of the compound of Formula VI;
c) reacting the compound of Formula VII with a chiral center-inverting base in a chiral center-inverting solvent to form a compound of Formula VIII: 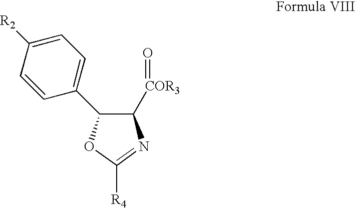
wherein R₂, R₃ and R₄ are as defined above and an inverted relative stereochemistry exists at the asymmetric α-carbonyl carbon compared to that of the compound of Formula VII; and
d) reacting the compound of Formula VIII with a reducing agent in a reducing-promoting solvent to form a compound of Formula IV: 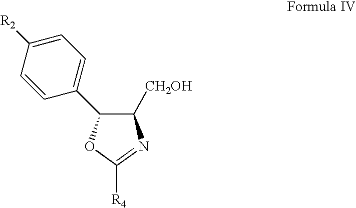
wherein R₂ and R₄ are as defined above.

2. The process of claim 1, wherein R₂ is methylthio, methylsulfoxy, or methylsulfonyl.

3. The process of claim 2, wherein R₂ is methylsulfonyl.

4. The process of claim 1, wherein R₃ is methyl, ethyl, propyl, isopropyl, butyl, iso-butyl or pentyl.

5. The process of claim 4, wherein R₃ is methyl or ethyl.

6. The process of claim 5, wherein R₃ is ethyl.

7. The process of claim 6, wherein R₄ is CH₂Cl, CHCl₂, CCl₃, CH₂Br, CHBr₂, CBr₃ CH₂F, CHF₂, or CF₃.

8. The process of claim 7, wherein R₄ is CH₂Cl, CHCl₂ or CCl₃.

9. The process of claim 8, wherein R₄ is CHCl₂.

10. The process of claim 1, wherein the compound of Formula V is a compound of Formula Va or an acid addition salt thereof: 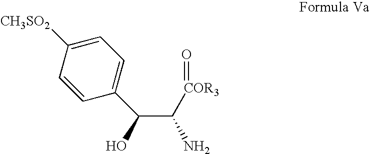
wherein R₃ is as defined above.

11. The process of claim 10, wherein the compound of Formula Va is the acid addition salt.

12. The process of claim 11, wherein the acid addition salt is a HCl salt.

13. The process of claim 1, wherein the compound of Formula V is a compound of Formula Vb or an acid addition salt thereof:

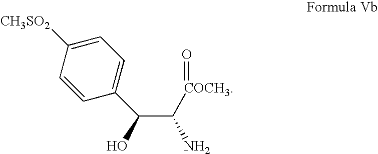

14. The process of claim 13, wherein the compound of Formula Vb is the acid addition salt.

15. The process of claim 14, wherein the acid addition salt is a HCl salt.

16. The process of claim 1, wherein the compound of Formula V is a compound of Formula Vc or the acid addition salt thereof:

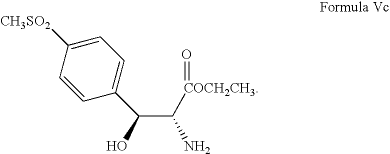

17. The process of claim 16, wherein the compound of Formula Vc is the acid addition salt.

18. The process of claim 14, wherein the acid addition salt is a HCl salt.

19. The process of claim 1, wherein the amide-promoting reagent of step a) has a formula of R₅COR₄, wherein R₄ is as defined above and R₅ is halo or C₁₋₆ alkoxy.

20. The process of claim 19, wherein R₄ is CH₂Cl, CHCl₂, CCl₃, CH₂Br, CHBr₂, CBr₃, CH₂F, CHF₂, or CF₃ and R₅ is C₁ or CH₃O.

21. The process of claim 20, wherein R₄ is CH₂Cl, CHCl₂ or CCl₃.

22. The process of claim 21, wherein R₄ is CHCl₂.

23. The process of claim 19, wherein the amide-promoting reagent is CH₃OCOCHCl₂ or ClCOCHCl₂.

24. The process of claim 23, wherein the amide-promoting reagent is ClCOCHCl₂.

25. The process of claim 23 or claim 24, wherein Florfenicol is the end-product.

26. The process of claim 1, wherein an amide-forming solvent of step a) comprises methanol, ethanol, propanol, isopropanol, acetone, methylene chloride, ethyl acetate, tetrahydrofuran, ether, toluene or a mixture thereof.

27. The process of claim 26, wherein the amide-forming solvent comprises methanol, ethanol, methylene chloride or a mixture thereof.

28. The process of claim 1, wherein an amide-promoting compound of step a) comprises potassium carbonate, potassium bicarbonate, sodium carbonate, sodium bicarbonate, trimethylamine triethylamine, p-toluene sulfonic acid, methanesulfonic acid, acetic acid, hydrochloric acid, sulfuric acid, nitric acid, phosphoric acid or a mixture thereof.

29. The process of claim 28, wherein the amide-promoting compound comprises triethylamine.

30. The process of claim 1, wherein the amide-promoting reagent of step a) and the compound of Formula V have a molar ratio between about 1:1 and about 3:1.

31. The process of claim 30, wherein the amide-promoting reagent is ClCOCHCl₂ and the molar ratio of ClCOCHCl₂ to the compound of Formula V is between about 1.2 and about 1.5 to about 1.

32. The process of claim 30, wherein the amide-promoting compound is triethylamine and the molar ratio of triethylamine to the compound of Formula V is between about 1.2 and about 1.5 to about 1.

33. The process of claim 30, wherein the amide-promoting compound is triethylamine and the molar ratio of triethylamine to an acid addition salt of the compound of Formula V is between about 2:1 and about 5:1.

34. The process of claim 1, wherein step a) has a temperature between about minus 25° C. to about 25° C.

35. The process of claim 30, wherein step a) has a temperature between about 0° C. to about 10° C.

36. The process of claim 1, wherein the compound of Formula VI is a compound of Formula VIa: 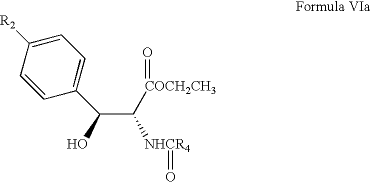
wherein R₂ and R₄ are as defined above.

37. The process of claim 1, wherein the compound of Formula VI is a compound of Formula VIb: 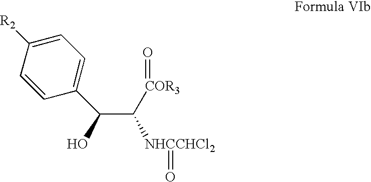
wherein R₂ and R₃ are as defined above.

38. The process of claim 1, wherein the compound of Formula VI is a compound of Formula VIc: 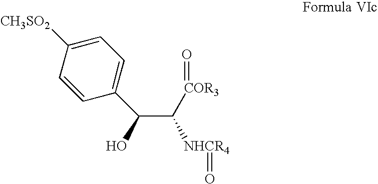
wherein R₃ and R₄ are as defined above.

39. The process of claim 1, wherein the compound of Formula VI is a compound of Formula VId: 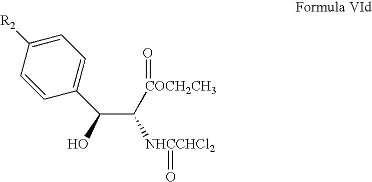
wherein R₂ is as defined above.

40. The process of claim 1, wherein the compound of Formula VI is a compound of Formula VIe: 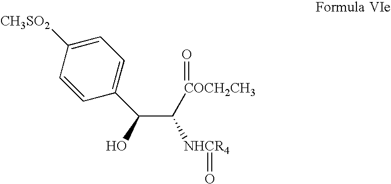
wherein R₄ is as defined above.

41. The process of claim 1, wherein the compound of Formula VI is a compound of Formula VIf: 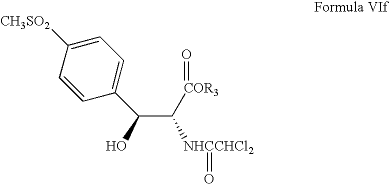
wherein R₃ is as defined above.

42. The process of claim 1, wherein the compound of Formula VI is a compound of Formula VIg:

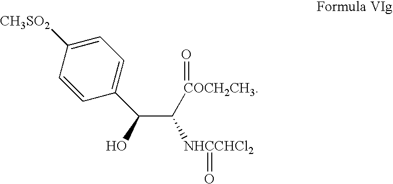

43. The process of claim 42, wherein Florfenicol is the end-product.

44. The process of claim 1, wherein the oxazoline-promoting reagent of step b) comprises thiony chloride, phosphorous trichloride, phosphorous pentachloride, phosphorous tribromide, phosphorous triiodide, phosphorous oxychloride, p-toluenesulfonyl chloride, p-bromosulfonyl chloride, p-nitrobenzenesulfonyl chloride, methanesulfonyl chloride, trifluoromethanesulfonyl chloride, nonafluorobutanesulfonyl chloride, 2,2,2-trifluoroethanesulfonyl chloride, or a mixture thereof.

45. The process of claim 44, wherein the compound of Formula VI and the oxazoline-promoting reagent of step b) form a compound of Formula VII: 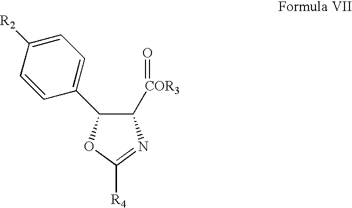
wherein R₂, R₃ and R₄ are as defined above and an inverted relative stereochemistry exists at the asymmetric benzylic carbon compared to that of the compound of Formula VI.

46. The process of claim 45, wherein the oxazoline-promoting reagent comprises thiony chloride.

47. The process of claim 45, wherein Florfenicol is the end-product.

48. The process of claim 1, wherein the oxazoline-forming solvent of step b) comprises methanol, ethanol, propanol, isopropanol, acetone, 1,2-dichloroethane, methylene chloride, chloroform, ethyl acetate, tetrahydrofuran, ether, toluene or a mixture thereof.

49. The process of claim 48, wherein the oxazoline-forming solvent comprises methylene chloride, chloroform or a mixture thereof.

50. The process of claim 1, wherein the oxazoline-promoting compound of step b) comprises sodium carbonate, potassium carbonate, sodium bicarbonate, potassium bicarbonate, sodium hydroxide, potassium hydroxide, 1,4-diazabicyclo[2.2.2]octane, pyridine, trimethylamine, triethylamine or a mixture thereof.

51. The process of claim 1, wherein the oxazoline-promoting reagent of step b) and the compound of Formula VI have a molar ratio between about 1:1 and about 6:1.

52. The process of claim 51, wherein the molar ratio is about 2:1.

53. The process of claim 51, wherein the oxazoline-promoting compound comprises triethylamine and the molar ratio of triethylamine to the oxazoline-promoting reagent is between about 1:1 and 3:1.

54. The process of claim 53, wherein the molar ratio is about 2:1.

55. The process of claim 1, wherein step b) has a temperature between about minus 25° C. and about 25° C.

56. The process of claim 54, wherein the temperature is between about 0° C. and about 10° C.

57. The process of claim 1, wherein the compound of Formula VII is a compound of Formula VIIIa: 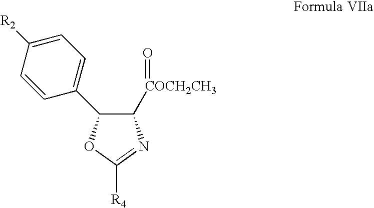
wherein R₂ and R₄ are as defined above.

58. The process of claim 1, wherein the compound of Formula VII is a compound of Formula VIIb: 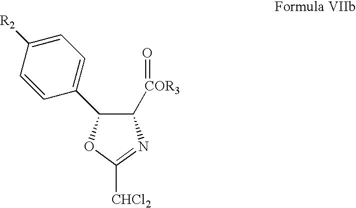
wherein R₂ and R₃ are as defined above.

59. The process of claim 1, wherein the compound of Formula VII is a compound of Formula VIIc: 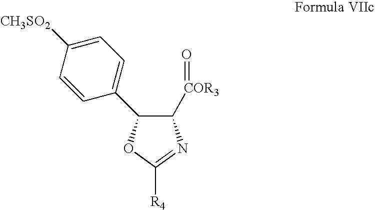
wherein R₃ and R₄ are as defined above.

60. The process of claim 1, wherein the compound of Formula VII is a compound of Formula VIId: 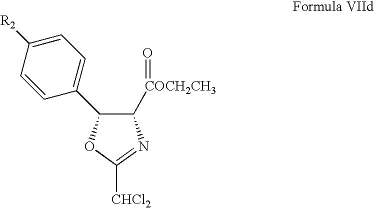
wherein R₂ is as defined above.

61. The process of claim 1, wherein the compound of Formula VII is a compound of Formula VIIe: 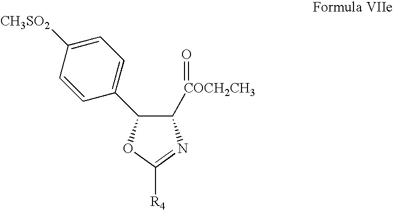
wherein R₄ is as defined above.

62. The process of claim 1, the compound of Formula VII is a compound of Formula VIIf: 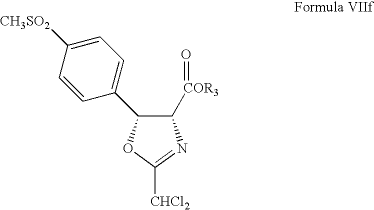
wherein R₃ is as defined above.

63. The process of claim 1, wherein the compound of Formula VII is a compound of Formula VIIg:

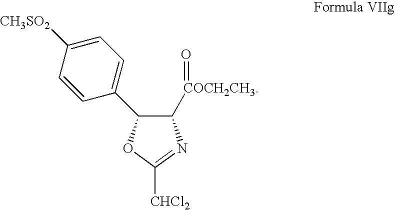

64. The process of claim 63, wherein Florfenicol is the end-product.

65. The process of claim 1, wherein the chiral center-inverting base of step c) comprises sodium methoxide, sodium ethoxide, potassium methoxide, potassium ethoxide, sodium hydroxide, potassium hydroxide or a mixture thereof.

66. The process of claim 1, wherein the compound of Formula VII and the chiral center-inverting base of step c) form a compound of Formula VIII: 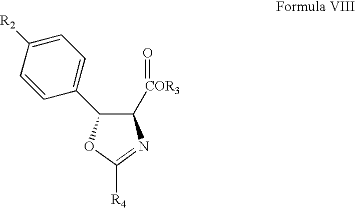
wherein R₂, R₃ and R₄ are as defined above and an inverted relative stereochemistry exists at the asymmetric α-carbonyl carbon compared to that of the compound of Formula VII.

67. The process of claim 1, wherein the chiral center-inverting solvent of step c) comprises methanol, ethanol, propanol, isopropanol, acetone, methylene chloride, ethyl acetate, tetrahydrofuran, ether, toluene or a mixture thereof.

68. The process of claim 66, wherein the chiral center-inverting solvent comprises methanol, ethanol, methylene chloride or a mixture thereof.

69. The process of claim 1, wherein the compound of Formula VIII is a compound of Formula VIIIa: 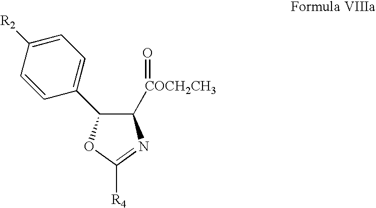
wherein R₂ and R₄ are as defined above.

70. The process of claim 1, wherein the compound of Formula VIII is a compound of Formula VIIIb: 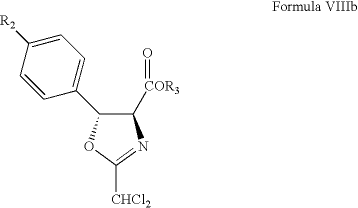
wherein R₂ and R₃ are as defined above.

71. The process of claim 1, wherein the compound of Formula VIII is a compound of Formula VIIIc: 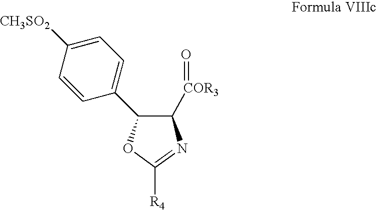
wherein R₃ and R₄ are as defined above.

72. The process of claim 1, wherein the compound of Formula VIII is a compound of Formula VIIId: 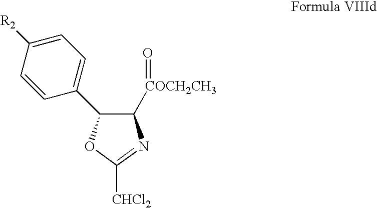
wherein R₂ is as defined above.

73. The process of claim 1, wherein the compound of Formula VIII is a compound of Formula VIIIe: 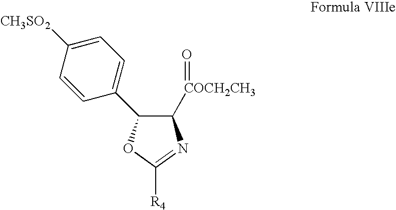
wherein R₄ is as defined above.

74. The process of claim 1, wherein the compound of Formula VIII is a compound of Formula VIIIf: 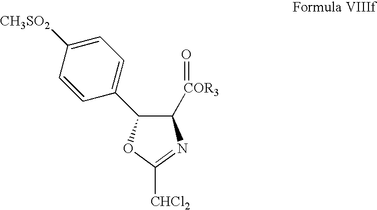
wherein R₃ is as defined above.

75. The process of claim 1, wherein the compound of Formula VIII is a compound of Formula VIIIg:

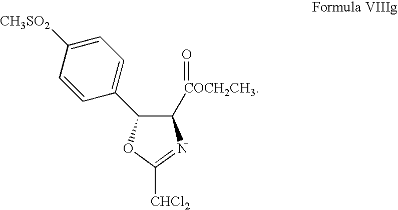

76. The process of claim 75, wherein Florfenicol is the end-product.

77. The process of claim 1, wherein the compound of Formula VIII and the reducing agent of step d) in a reducing-promoting solvent form a compound of Formula IV: 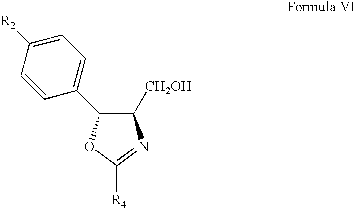
wherein R₂ and R₄ are as defined above.

78. The process of claim 77, wherein the reducing agent comprises NaBH₄, KBH₄, Ca(BH₄)₂, LiBH₄ or a mixture thereof.

79. The process of claim 78, wherein the reducing agent comprises KBH₄, NaBH₄ or a mixture thereof.

80. The process of claim 79, wherein the reducing agent comprises KBH₄.

81. The process of claim 77, wherein the reducing-promoting solvent comprises water, methanol, ethanol, propanol, isopropanol, butanol, pentanol and a mixture thereof.

82. The process of claim 81, wherein the reducing-promoting solvent comprises water, methanol, ethanol or a mixture thereof.

83. The process of claim 82, wherein the reducing-promoting solvent comprises methanol.

84. The process of claim 77, wherein the reducing agent of step d) and the compound of Formula VIII have a molar ratio between about 1:1 and about 2:1.

85. The process of claim 84, wherein the reducing agent is KBH₄, and the molar ratio of KBH₄ to the compound of Formula VIII is about 1.5:1.

86. The process of claim 77, wherein the reducing-promoting solvent comprises methanol.

87. The process of claim 77, wherein the reacting step d) is carried out at a temperature of about 30° C. to about 80° C. in about 8 hours.

88. The process of claim 87, wherein the temperature is less than about 60° C. and the step b) is substantially complete in less than about 6 hours.

89. The process of claim 1, wherein the reducing agent comprises LiAlH₄, NaAlH₄, or a mixture thereof.

90. The process of claim 89, wherein the reducing-promoting solvent comprises ether, tetrahydrofuran or a mixture thereof.

91. The process of claim 1, wherein the compound of Formula IV is a compound of Formula IVa: 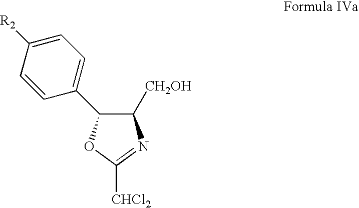
wherein R₂ is as defined above.

92. The process of claim 1, wherein the compound of Formula IV is a compound of Formula IVb: 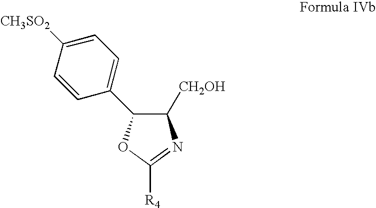
wherein R₄ is as defined above.

93. The process of claim 1, wherein the compound of Formula IV is a compound of Formula IVc:

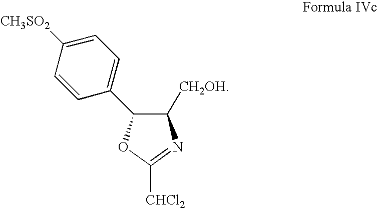

94. The process of claim 93, wherein Florfenicol is the end-product.

95. The process of claim 1, further comprising the step of fluorinating the compound of Formula IV with a fluorinating agent in the presence of an organic solvent to obtain the compound of Formula IX: 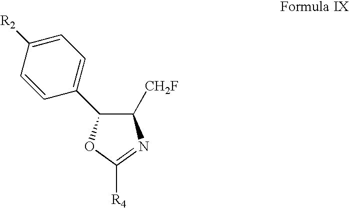
wherein R₂ and R₄ are as defined above.

96. The process of claim 95, wherein the fluorinating agent comprises sodium fluoride, potassium fluoride, cesium fluoride, tetrabutylammonium fluoride, 1,1,2,2,3,3,4,4,4-nonafluoro-1-butanesulfonyl fluoride, chloromethyl-4-fluoro-1,4-diazoniabicyclo[2.2.2]octane bis-(tetrafluoroborate), N-(2-chloro-1,1,2-trifluoroethyl)diethylamine, N-(2-chloro-1,1,2-trifluoroethyl)dimethylamine, N-(2-chloro-1,1,2-trifluoroethyl)dipropylamine, N-(2-chloro-1,1,2-trifluoroethyl)pyrrolidine, N-(2-chloro-1,1,2-trifluoroethyl)-2-methylpyrrolidine, N-(2-chloro-1,1,2-trifluoroethyl)-4-methylpiperazine, N-(2-chloro-1,1,2-trifluoroethyl)-morpholine, N-(2-chloro-1,1,2-trifluoroethyl)piperidine, 1,1,2,2-tetrafluoroethyl-N,N-dimethylamine, (Diethylamino) sulfur trifluoride, Bis-(2-methoxyethyl)aminosulfur trifluoride, N,N-diethyl-1,1,2,3,3,3-hexafluoro-1-propanamine, or a mixture thereof.

97. The process according to claims 96, wherein the fluorinating agent is N,N-diethyl-1,1,2,3,3,3-hexafluoro-1-propanamine.

98. The process of claim 96, wherein the fluorinating agent and the compound of Formula IV have a molar ratio between about 1:1 and about 2:1.

99. The process according to claim 98, wherein the fluorinating agent is N,N-diethyl-1,1,2,3,3,3-hexafluoro-1-propanamine.

100. The process of claim 99, wherein the molar ratio of the N,N-diethyl-1,1,2,3,3,3-hexafluoro-1-propanamine to the compound of Formula IV is about 1.5:1.

101. The process of claim 95, wherein the fluorinating step is carried out at a temperature of about 80° C. to about 110° C. and at a pressure of about 60 psi.

102. The process of claim 95, wherein the organic solvent of the fluorinating step comprises 1,2-dichloroethane, methylene chloride, chloroform, chlorobenzene, a chlorinated hydrocarbon, or a mixture thereof.

103. The process of claim 95, wherein the organic solvent comprises methylene chloride.

104. The process of claim 95, wherein the compound of Formula IX corresponds to a compound of Formula IXa: 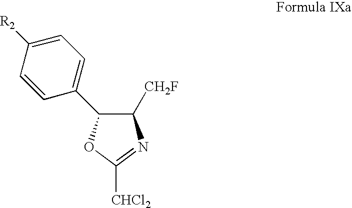
wherein R₂ is as defined above.

105. The process of claim 95, wherein the compound of Formula IX corresponds to a compound of Formula IXb: 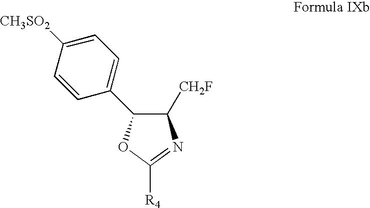
wherein R₄ is as defined above.

106. The process of claim 95, wherein the compound of Formula IX corresponds to a compound of Formula IXc:

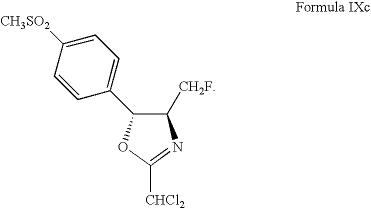

107. The process of claim 95, further comprising the step of hydrolyzing the compound of Formula IX with an acid catalyst or a basic catalyst and water to form a compound of Formula X: 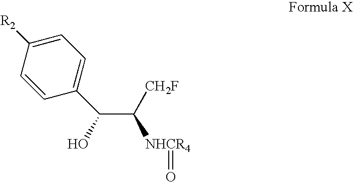
wherein R₁ and R₄ are as defined above.

108. The process of claim 107, wherein the compound of Formula X is Florfenicol.

109. The process of claim 107, wherein the acid catalyst comprises at least one inorganic acid, at least one organic acid or a mixture thereof.

110. The process of claim 108, wherein the acid catalyst comprises a dilute aqueous hydrochloric acid, sulfuric acid, nitric acid, phosphoric acid, acetic acid, trifluoroacetic acid, methanesulfonic acid, p-toluene sulfonic acid, or a mixture thereof.

111. The process of claim 110, wherein the acid catalyst comprises p-toluene sulfonic acid.

112. The process of claim 107, wherein the basic catalyst comprises at least one inorganic base, at least one organic base or a mixture thereof.

113. The process of claim 112, wherein the basic catalyst comprises LiOH, NaOH, KOH, Li₂CO₃, Na₂CO₃, K₂CO₃, NH₄OH sodium methoxide, sodium ethoxide, potassium methoxide, potassium ethoxide or a mixture thereof.

114. The process of claim 112, wherein the basic catalyst comprises NH₄OH.

115. The process of claim 107, wherein the hydrolyzing step is carried out at a temperature less than or equal to about 100° C.

116. The process of claim 115, wherein the temperature is less than about 30° C.

117. The process of claim 107, wherein the hydrolyzing step further comprises heating the compound of Formula IX with the acid catalyst or the basic catalyst and water at a temperature less than about 100° C.

118. The process of claim 107, wherein about 0.5 to about 3 molar equivalents of water are used for each mole of the compound of Formula IX.

119. The process of claim 107, wherein about 1 to about 2 molar equivalents of water are used for each mole of the compound of Formula IX.

120. The process of claim 1, wherein the compound of Formula X corresponds to a compound of Formula Xa: 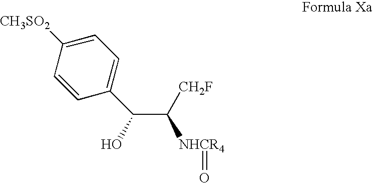
wherein R₄ is as described above.

121. The process of claim 1, wherein the compound of Formula X corresponds to a compound of Formula Xb: 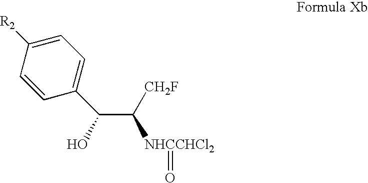
wherein R₂ is as described above.

122. The process of claim 120, wherein the compound of Formula X is Florfenicol.

123. The process of claim 120, further comprising the step of purifying the compound of Formula X to obtain purified compound of Formula X.

124. The process of claim 123, wherein the purifying step comprises using a mixture of a C₁₋₁₀ alkyl monoalcohol, a C₁₋₁₀ alkyl dialcohol or a C₁₋₁₀ alkyl trialcohol and water.

125. The process of claim 124, wherein the mixture comprises methanol, ethanol, propanol, iso-propanol, butanol, sec-butanol, t-butanol, pentanol, ethylene glycol, propylene glycol, butylene glycol, glycerin, or a mixture thereof and water.

126. The process of claim 125, wherein the mixture comprises isopropanol and water.

127. The process of claim 126, wherein the isopropanol and water mixture has a ratio between about 1:5 and about 5:1

128. The process of claim 127, wherein the ratio of isopropanol to water is about 1:1.

129. The process of claim 128, wherein the compound of Formula X and the about 1:1 isopropanol and water mixture have a weight to volume ratio between about 1:1 and about 10:1.

130. The process of claim 129, wherein the weight to volume ratio of the compound of Formula IX to the about 1:1 isopropanol and water mixture is about 1:4.6.

131. The process of claim 130, wherein the purifying step has a dissolution temperature that is the reflux point of the 1:1 isopropanol and water mixture.

132. The process of claim 123, wherein the purifying step comprises using a cooling temperature of about 10° C. to about 30° C. to obtain crystallized compound of Formula X.

133. The process of claim 132, wherein the cooling temperature is about 20° C. to about 25° C.

134. The process of claim 132, wherein the compound of Formula X is Florfenicol.

135. A compound of Formula V or an acid addition salt thereof: 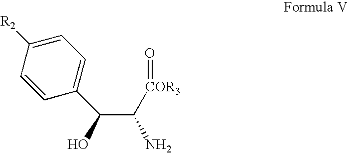
wherein R₂ and R₃ are as defined above, with the provisos that:
if R₂ is methylsulfonyl, then R₃ is not CH₃ or CH₂CH₃; and
if the compound of Formula V is the acid addition salt, then the acid addition salt is the HCl, HNO₃, H₂SO₄, H₃PO₄, or acetic acid salt.

136. A compound of Formula VI or an acid addition salt thereof: 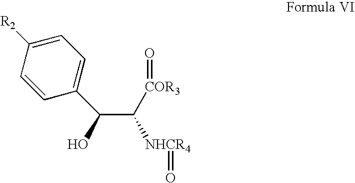
wherein R₂ is methylsulfonyl; R₃ is CH₃ or CH₂CH₃; and R₄ is CH₂Cl, CHCl₂, CCl₃, CH₂Br, CHBr₂, CBr₃, CH₂F, CHF₂, or CF₃, with the proviso that if the compound of Formula VI is the acid addition salt, then the acid addition salt is the HCl, HNO₃, H₂SO₄, H₃PO₄, or acetic acid salt.

137. The compound of claim 136, wherein; R₃ is CH₃ or CH₂CH₃; and R₄ is CHCl₂.

138. A compound of Formula VII or an acid addition salt thereof: 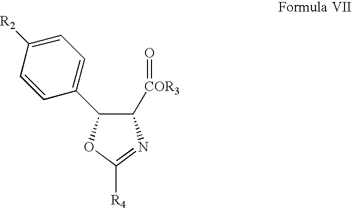
wherein R₂, R₃ and R₄ are as defined above, with the provisos that:
if R₂ is methylsulfonyl and R₄ is phenyl, then R₃ is not CH₃ or CH₂CH₃; and
if the compound of Formula VI is the acid addition salt, then the acid addition salt is the HCl, HNO₃₁H₂SO₄, H₃PO₄, or acetic acid salt.

139. The compound of claim 138, wherein R₂ is methylsulfonyl, R₃ is CH₃ or CH₂CH₃; and R₄ is CHCl₂.

140. A compound of Formula VIII or an acid addition salt thereof: 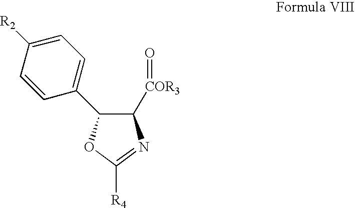
wherein R₂, R₃ and R₄ are as defined above, with the provisos that:
if R₂ is methylsulfonyl and R₄ is phenyl, then R₃ is not CH₃ or CH₂CH₃; and
if the compound of Formula VI is the acid addition salt, then the acid addition salt is the HCl, HNO₃, H₂SO₄, H₃PO₄, or acetic acid salt.

141. The compound of claim 140, wherein R₂ is methylsulfonyl, R₃ is CH₃ or CH₂CH₃; and R₄ is CHCl₂.
